(19) United States
(12) Patent Application Publication
(10) Pub. No.: US 2012/0296102 A1
(43) Pub. Date: Nov. 22, 2012

(54) METHOD FOR PRODUCING PROPYLENE OXIDE

(75) Inventors: Tomonori Kawabata, Toyonaka-shi (JP); Hiroaki Abekawa, Toyonaka-shi (JP)

(73) Assignee: SUMITOMO CHEMICAL COMPANY, LIMITED, Chuo-ku, Tokyo (JP)

(21) Appl. No.: 13/576,319

(22) PCT Filed: Jan. 27, 2011
(86) PCT No.: PCT/JP2011/052205

§ 371 Date: Jul. 31, 2012

(30) Foreign Application Priority Data
Feb. 3, 2010 (JP) 2010-021978

(51) Int. Cl. C07D 301/06

(52) U.S. Cl. 549/533

(57) ABSTRACT

It is intended to provide a production method for producing propylene oxide from propylene, hydrogen and oxygen, with improved reaction rate. The present invention provides a method for producing propylene oxide, comprising a step of reacting propylene, hydrogen and oxygen, in the presence of a Pd-supported catalyst, a titanosilicate catalyst and a Pd-free carbon material, in a liquid phase.

### BACKGROUND ART

A production method comprising a step of reacting propylene, oxygen and hydrogen in the presence of a noble metal-supported catalyst and a titanosilicate catalyst is known as a method for producing propylene oxide (see e.g., Non Patent Literature 1).

On the other hand, a method for producing propylene oxide with high efficiency is preferable for industry.

### CITATION LIST

### Non Patent Literature

Non Patent Literature 1: Applied Catalysis A: General 213, (2001), 163-171

### SUMMARY OF INVENTION

### Technical Problem

A method for producing propylene oxide efficiently from propylene, oxygen and hydrogen has been demanded.

The present inventors have conducted diligent studies to solve the problem and consequently reached the present invention.

Specifically, the present invention provides:

[1] a method for producing propylene oxide, comprising a step of reacting propylene, hydrogen and oxygen, in the presence of a Pd-supported catalyst, a titanosilicate catalyst and a Pd-free carbon material, in a liquid phase;
[2] the method according to [1], wherein the Pd-supported catalyst consists of a carrier and Pd supported by the carrier, and the Pd-free carbon material does not form the carrier of the Pd-supported catalyst;
[3] the method according to [1], wherein the Pd-supported catalyst comprises at least one carrier selected from the group consisting of silica, alumina, active carbon and carbon black;
[4] the method according to [1], wherein the Pd-supported catalyst comprises a carrier selected from the group consisting of active carbon and carbon black;
[5] the method according to any one of [1] to [4], wherein the Pd-free carbon material is active carbon, carbon black or a mixture thereof;
[6] the method according to any one of [1] to [4], wherein the Pd-free carbon material is active carbon;
[7] the method according to any one of [1] to [6], wherein the step comprises reacting propylene, hydrogen and oxygen, further in the presence of a polycyclic compound having 2 to 30 rings, in a liquid phase;
[8] the method according to [7], wherein the polycyclic compound is a condensed polycyclic aromatic compound; and
[9] the method according to [7], wherein the polycyclic compound comprises anthraquinone.

### Advantageous Effects of Invention

According to the present invention, a propylene oxide can be produced from propylene, hydrogen and oxygen, at improved production rate.

### BRIEF DESCRIPTION OF DRAWINGS

FIG. 1 is an X-ray diffraction pattern of Ti-MWW precursor A.

FIG. 2 is an UV-visible absorption spectrum of Ti-MWW precursor A.

FIG. 3 is an UV-visible absorption spectrum of titanosilicate B.

### DESCRIPTION OF EMBODIMENTS

A production method of the present invention comprises a step of reacting propylene, hydrogen and oxygen, in the presence of a Pd-supported catalyst, a titanosilicate catalyst and a Pd-free carbon material, in a liquid phase. Hereinafter, referring to this step as “the present step” and the reaction of propylene, hydrogen and oxygen as “the present reaction”, specific embodiments of the present invention will be described.

<Pd-Free Carbon Material>

The Pd-free carbon material used in the present step means a carbon material that does not substantially contain Pd (palladium). In this context, “not substantially contain Pd” means that a Pd content percentage by weight (hereinafter, referred to as a “Pd content”) is lower than 0.01% by weight, and the carbon material means a material composed mainly of carbon. Examples of such a Pd-free carbon material include: active carbon; carbon black; carbon nanotube; mesoporous carbon; carbon fiber; fullerene or fullerene analog compounds such as C70; graphite; and diamond. While the Pd-free carbon materials mentioned above differ in name depending on shape, crystal form, or the like, all are composed mainly of carbon. Moreover, the carbon materials mentioned above all have the advantage that those having a Pd content of lower than 0.01% by weight are easily available from the market. Moreover, the commercially available Pd-free carbon material can also be subjected to the present invention, after confirming by an appropriate analysis method such as fluorescence X-ray analysis, e.g. fundamental parameter (FP) method, or IPC emission analysis (analysis method whose lower analysis limit of the Pd content is lower than 0.01% by weight) that its Pd content is lower than 0.01% by weight.

In the present invention, the Pd-free carbon material does not support Pd; it forms a particle essentially consisting of carbon atoms. This particle is independent from Pd-supported catalyst in the liquid phase.

The Pd-free carbon material may be activated by oxidation or the like. In case the Pd-free carbon material is activated, it is possible to obtain the propylene oxide more efficiently. Examples of methods for this activation (activation methods) include:

a method comprising contacting the Pd-free carbon material with water vapor for activation at a temperature condition of 750° C. or higher;

a method comprising contacting the Pd-free carbon material with carbon dioxide for activation at a temperature condition of 850 to 1100° C.;

a method comprising contacting the Pd-free carbon material with oxidizing gas such as oxygen-containing gas; and

activation methods using chemicals such as zinc chloride, phosphoric acid, sulfuric acid, nitric acid, calcium chloride and sodium hydroxide.

Here, a specific activation method will be described. For example, in the case of using diamond as the Pd-free carbon material, a method comprising air-oxidizing commercially available fine diamond powder for polishing at a temperature condition of 450° C. for approximately 1 hour to form oxidized diamond, is known (this activation method is described in Japanese Patent Laid-Open No. 2002-177783). Moreover, for example, in the case of using active carbon derived from plants such as sawdust or palm husks as the Pd-free carbon material, the material may be more activated by a method comprising contacting a carbon material with water vapor for activation at a temperature condition of 750 to 900° C., a method comprising contacting the carbon material with zinc chloride for activation at a temperature condition of 600 to 750° C., or the like.

Industrially, it is preferred that the Pd-free carbon material used in the present step should be inexpensive. In terms of being inexpensive, a Pd-free carbon material selected from the group consisting of active carbon, carbon black and graphite is preferable; active carbon, carbon black or a mixture thereof is more preferable; and the active carbon is further preferable. Particularly, the active carbon is commercially available as one activated in advance with zinc chloride, water vapor, or the like and is preferable in terms of the easy availability.

Moreover, it is preferred that the Pd-free carbon material should be large in surface area (have a high surface area). With this high surface area as an index, the specific surface area (BET specific surface area) based on nitrogen gas adsorption is preferably 10 m²/g or higher, more preferably 50 m²/g or higher, even more preferably 100 m²/g or higher. Also in terms of the high surface area, examples of a preferable Pd-free carbon material can include active carbon and carbon black, and among them, the active carbon is a particularly preferable one. The BET specific surface area of commercially available active carbon or carbon black is generally 10 m²/g or higher, and, particularly, the active carbon is inexpensively commercially available as one having a surface area as very high as 1000 m²/g or higher in BET specific surface area. It is preferred to use a carbon material as the Pd-free carbon material in the present step after determining its BET specific surface area and confirming that the BET specific surface area is 10 m²/g or higher. Likewise, in the case of mixing plural kinds of Pd-free carbon materials for use, the types or mixing ratio of the Pd-free carbon materials to be mixed may be determined such that the BET specific surface area of the Pd-free carbon materials after mixing is 10 m²/g or higher. The upper limit of the BET specific surface area of the Pd-free carbon material is approximately 3000 m²/g in terms of the easy availability of materials. In the present specification, the BET specific surface area can be measured by micromeritics automatic surface area analyzer.

It is preferred that the amount of the Pd-free carbon material used in the present step should be determined in consideration of the amount of the Pd-supported catalyst used together therewith. Specifically, the weight ratio between the Pd-free carbon material and the Pd-supported catalyst is indicated in [Pd-free carbon material]/[Pd-supported catalyst] and is preferably in the range of 1/1 to 1000/1, more preferably in the range of 1/1 to 200/1. When the weight ratio is too small, the reaction time of the present reaction may be a long time because sufficient reaction activity is not obtained. When the weight ratio is too large, it is required to increase the size of a reactor used in the present step, by more than needed.

<Pd-Supported Catalyst>

The Pd-supported catalyst used in the present step is one in which Pd (palladium) is supported on a carrier, and is one having catalytic ability related to the present reaction. The carrier needs only to be one capable of supporting Pd, examples of which include: oxides such as silica, alumina, titania, zirconia and niobia; niobic acid, zirconic acid, tungstic acid and titanic acid; and carbon materials, and a mixture, mixed oxide, or the like of plural types selected therefrom can also be used. In this context, the mixed oxide is a crystalline aluminosilicate or the like. It is preferred that this carrier should be easily available in such a way that it is commercially available, and it is more preferred to be inexpensive. Examples of inexpensively commercially available carriers include niobic acid, active carbon, carbon black, silica gel, silica, alumina and aluminum-containing zeolite. Examples of commercially available aluminum-containing zeolites include zeolite A, zeolite X, zeolite Y, ZSM-5, zeolite T, zeolite P, zeolite L, zeolite beta, mordenite, ferrierite and chabazite. Among these aluminum-containing zeolites, there is one whose ion is exchanged using sodium ion, potassium ion, calcium ion, ammonium ion, or the like for compensating for lack of the electric charge of aluminum ion. Of those mentioned above, examples of more preferable carriers include those selected from the group consisting of silica, alumina, active carbon and carbon black; the active carbon or carbon black is more preferable; and the active carbon is particularly preferable.

The Pd-supported catalyst generally consists of the carrier as mentioned above and Pd supported by the carrier. On the other hand, the Pd-free carbon material not to use for the carrier exists independently from Pd-supported catalyst.

The Pd-supported catalyst can be prepared by supporting Pd onto the carrier. The supporting of Pd can be carried out according to a method known in the art. For example, the Pd-supported catalyst can be prepared by supporting a palladium compound (e.g., palladium chloride and tetraamminepalladium (II) chloride) as a Pd source onto the carrier by an impregnation method or the like and then reducing the supported palladium compound using a reducing agent such as hydrogen. In such a preparation method, for example, a temperature condition of 0 to 500° C. is adopted. Supporting the palladium compound on the carrier and/or reducing the palladium compound may be carried out in a gas phase or may be carried out in a liquid phase, and the temperature condition can be adjusted appropriately depending on the situation in which it is carried out in a gas phase or carried out in a liquid phase. The palladium in the palladium compound supported on the carrier has a positive charge, and the Pd-supported catalyst is prepared by reducing a portion or the whole thereof to zero-valent palladium. This reduction is carried out prior to subjecting it to the present step, and the Pd-supported catalyst may be prepared in advance. The palladium compound supported on a carrier is subjected to the present step, and reduction may be carried out in a reactor for carrying out the present step, to prepare the Pd-supported catalyst.

Moreover, examples of another form of Pd-supported catalyst preparation include a method using colloidal palladium as a palladium source. A method comprising first mixing colloidal palladium solution and the carrier to support the palladium onto the carrier, then filtering the mixture, and drying the filter cake is known as this method. Since the palladium contained in the colloidal palladium used here is already zero-valent, the Pd-supported catalyst can be prepared very conveniently by using commercially available colloidal palladium. The amount of Pd in the entire Pd-supported catalyst is, generally, in the range of 0.01 to 20% in mass and more preferably in the range of 0.1 to 5% in mass.

Although specific examples of the Pd-supported catalyst used in the present step and its preparation method are shown above, Pd in the Pd-supported catalyst may be pure Pd metal or may be Pd-containing alloy. Examples of a metal other than Pd in the alloy include a noble metal selected from the group consisting of platinum, ruthenium, gold, rhodium and iridium. Examples of alloys preferable for use in the Pd-supported catalyst include pallidum/platinum alloy and palladium/gold alloy.

<Titanosilicate Catalyst>

The titanosilicate catalyst is a titanosilicate having epoxidation ability for propylene. Hereinafter, the titanosilicate used as the titanosilicate catalyst will be described in detail.

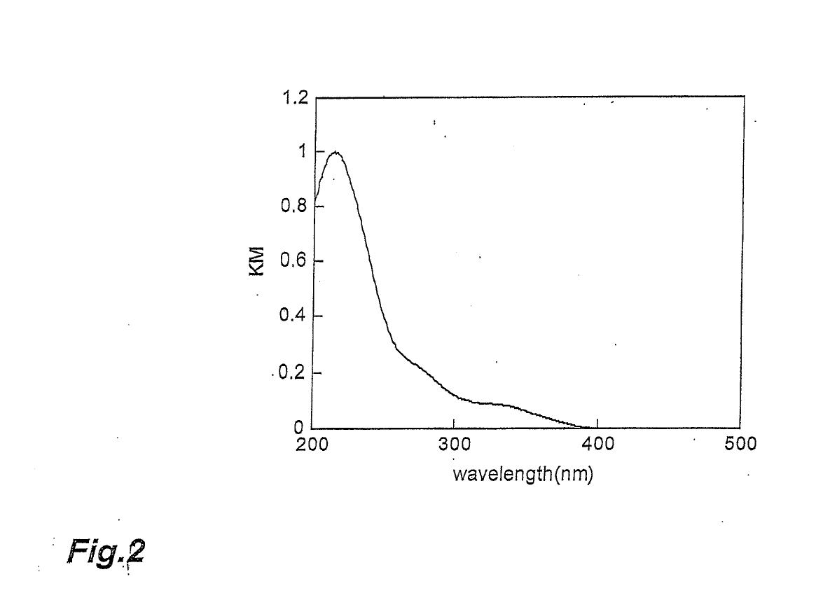

The titanosilicate is a generic name for silicate having tetracoordinated Ti (titanium atom) and is one having a porous structure. The titanosilicate constituting the titanosilicate catalyst means a titanosilicate substantially having tetracoordinated Ti and is one whose UV-visible absorption spectrum of a wavelength region of 200 nm to 400 nm has the greatest absorption peak in a wavelength region of 210 nm to 230 nm (see e.g., FIGS. 2(d) and 2(e) in Chemical Communications, 1026-1027, (2002)). This UV-visible absorption spectrum can be measured by a diffuse reflection method using an UV-visible spectrophotometer equipped with a diffuse reflection attachment.

The titanosilicate used as the titanosilicate catalyst is preferably one having a pore composed of 10- or more membered oxygen ring, in terms of having high epoxidation ability for propylene.

When the pore is too small, the contact between the raw materials of reaction (propylene, etc.) placed in the pore and active sites in the pore may be inhibited, or the mass transfer of the raw materials of reaction in the pore may be limited. In this context, the pore means one composed of Si—O or Ti—O bonds. The pores may be hemispherical pores called side pockets, and the pores do not have to penetrate a primary particle of the titanosilicate. Moreover, the “10- or more membered oxygen ring” means that the ring structure has 10 or more oxygen atoms in either (a) the section of the narrowest place in the pores or (b) the entrance to the pores. The pore composed of a 10- or more membered oxygen ring in the titanosilicate can generally be confirmed by the analysis of an X-ray diffraction pattern. Moreover, if the titanosilicate has a known structure, it can be confirmed conveniently by comparing the X-ray diffraction pattern with a known one.

Examples of the titanosilicate used as the titanosilicate catalyst include titanosilicates described in 1 to 7 below.

1. Crystalline titanosilicate having pores composed of 10-membered oxygen ring;
TS-1 having an MFI structure represented by the structural code specified by the International Zeolite Association (IZA) (e.g., U.S. Pat. No. 4,410,501), TS-2 having an MEL structure (e.g., Journal of Catalysis 130, 440-446, (1991)), Ti-ZSM-48 having an MRE structure (e.g., Zeolites 15, 164-170, (1995)), Ti-FER having an FER structure (e.g., Journal of Materials Chemistry 8, 1685-1686 (1998)), etc.
2. Crystalline titanosilicate having pores composed of 12-membered oxygen ring;
Ti-Beta having a BEA structure (e.g., Journal of Catalysis 199,41-47, (2001)), Ti-ZSM-12 having an MTW structure (e.g., Zeolites 15, 236-242, (1995)), Ti-MOR having an MOR structure (e.g., The Journal of Physical Chemistry B 102, 9297-9303, (1998)), Ti-ITQ-7 having an ISV structure (e.g., Chemical Communications 761-762, (2000)), Ti-MCM-68 having an MSE structure (e.g., Chemical Communications 6224-6226, (2008)), Ti-MWW having an MWW structure (e.g., Chemistry Letters 774-775, (2000)), etc.
3. Crystalline titanosilicate having pores composed of 14-membered oxygen ring;
Ti-UTD-1 having a DON structure (e.g., Studies in Surface Science and Catalysis 15, 519-525, (1995)), etc.
4. Laminar titanosilicate having pores composed of 10-membered oxygen ring;

### Ti-ITQ-6 (e.g., Angewandte Chemie International Edition 39, 1499-1501, (2000)), etc.

5. Laminar titanosilicate having pores composed of 12-membered oxygen ring;
Ti-MWW precursor (e.g., EP Patent Publication No. 1731515A1), Ti-YNU-1 (e.g., Angewandte Chemie International Edition 43, 236-240, (2004)), Ti-MCM-36 (e.g., Catalysis Letters 113, 160-164, (2007)), Ti-MCM-56 (e.g., Microporous and Mesoporous Materials 113, 435-444, (2008)), etc.
6. Mesoporous titanosilicate;

Ti-MCM-41 (e.g., Microporous Materials 10, 259-271, (1997)), Ti-MCM-48 (e.g., Chemical Communications 145-146, (1996)), Ti-SBA-15 (e.g., Chemistry of Materials 14, 1657-1664, (2002)), etc.

7. Silylated titanosilicate;

Compounds in which any of the titanosilicates described in 1 to 4 above is silylated, such as silylated Ti-MWW.

The “12-membered oxygen ring” means a ring structure whose number of oxygen atoms is 12 in the position (a) or (b) already described in the description of the 10-membered oxygen ring. Likewise, the “14-membered oxygen ring” means a ring structure whose number of oxygen atoms is 14 in the position (a) or (b).

The titanosilicate encompasses titanosilicates having a laminar structure, such as a laminar precursor of a crystalline titanosilicate and a titanosilicate having the expanded distance between the layers of a crystalline titanosilicate. The laminar structure can be confirmed by electronic microscopic observation or the measurement of the X-ray diffraction pattern. The laminar precursor means, for example, a titanosilicate that forms a crystalline titanosilicate by performing treatment such as dehydration condensation. The pore composed of a 12- or more membered oxygen ring in the laminar titanosilicate can be confirmed easily from the structure of the corresponding crystalline titanosilicate.

Moreover, the titanosilicates 1 to 5 and 7 have pores of 0.5 nm to 1.0 nm in pore size. This pore size means the longest size in (a) the section of the narrowest place in the pores or (c) the section of the widest place in the entrance of the pores and preferably means the diameter in this position. This pore size can be determined by the analysis of the X-ray diffraction pattern.

The mesoporous titanosilicate is a generic name for a titanosilicate having a regular mesopore. The regular mesopore means a structure in which mesopores are regularly and repeatedly arranged. The mesopore means a pore having a pore size of 2 nm to 10 nm.

The silylation of the titanosilicate can be carried out by contacting a silylating agent with the titanosilicate. Examples of the silylating agent include 1,1,1,3,3,3-hexamethyldisilazane and trimethylchlorosilane. The silylation with the silylating agent is described in, for example, EP Patent Publication No. EP1488853A1.

Although the titanosilicate used as the catalyst is described above in detail as to the titanosilicate catalyst used in the present step, those particularly preferred as the titanosilicate catalyst, among the titanosilicates 1 to 7, are Ti-MWW and a Ti-MWW precursor, further particularly preferably a Ti-MWW precursor. Of course, such Ti-MWW or a Ti-MWW precursor may be silylated and used in the titanosilicate catalyst, or the Ti-MWW or Ti-MWW precursor may be molded by a method known in the art and used in the titanosilicate catalyst.

<Method for Producing Propylene Oxide>

As described above, the present step comprises reacting propylene, hydrogen and oxygen in the presence of a Pd-supported catalyst, a titanosilicate catalyst and a Pd-free carbon material to obtain propylene oxide. By the action of the Pd-supported catalyst known as a hydrogen peroxide-synthesizing catalyst, hydrogen peroxide is first formed from hydrogen and oxygen, and the formed hydrogen peroxide reacts with propylene by the action of the titanosilicate catalyst to form propylene oxide.

The suitable Ti/Si mole ratio of titanosilicate catalyst for the present reaction is generally 0.001 to 0.1 and preferably 0.005 to 0.05.

The present reaction that forms propylene oxide proceeds in a liquid phase. Specifically, hydrogen, oxygen and propylene in a gas phase in a reactor are dissolved in a liquid phase, i.e., solvent, containing the Pd-supported catalyst, the titanosilicate catalyst and the Pd-free carbon material, hydrogen reacts with oxygen in the liquid phase to form hydrogen peroxide by the action of the Pd-supported catalyst, and this hydrogen peroxide reacts with propylene in the liquid phase to form propylene oxide by the action of the titanosilicate catalyst.

For example,
a method using a Pd-Pt (palladium/platinum alloy) catalyst supported by TS-1, in a methanol/water mixed solvent (e.g., Applied Catalysis A: General 213, 163-171, (2001));
a method using a Pd-supported catalyst (Pd supported by carbon black) and a titanosilicate catalyst composed of Ti-MWW or a Ti-MWW precursor, in an acetonitrile/water mixed solvent (e.g., WO2007/080995); and
a method using a Pd-supported catalyst (Pd supported by active carbon or niobic acid) and a titanosilicate catalyst composed of Ti-MWW, in an acetonitrile/water mixed solvent (e.g., WO2008/090997)
are known as the reaction that forms propylene oxide from propylene, hydrogen and oxygen by the action of the Pd-supported catalyst and the titanosilicate catalyst. However, in these documents, any mention was not made about the reaction in the present invention, i.e., the reaction which is conducted in the presence of the Pd-free carbon material added as the particles different from the Pd-supported catalyst. Such reaction is based on the present inventors' own findings. Even in the case of using a carbon material (e.g., active carbon) as the carrier of the Pd-supported catalyst, it is important to allow the Pd-free carbon material to coexist, aside from such a Pd-supported catalyst (Pd-supported carbon material).

Pd-free carbon material forms a particle substantially consisting of carbon atom. Therefore, even if a Pd-supported catalyst has a carbon, the Pd-free carbon material exists in another particle independently from the Pd-supported catalyst.

Specifically, enhancement of the reaction rate and extension of the Pd-supported catalyst longevity can be achieved favorably not by increasing the amount of the carbon with respect to the amount of Pd supported in the Pd-supported carbon material (decreasing the amount of Pd supported in the Pd-supported carbon material) but by allowing the Pd-free carbon material to coexist without changing the amount of the carbon used for the carrier of Pd. Such findings have also been obtained by the study of the present inventors.

The amount of the titanosilicate catalyst used can be adjusted depending on the form of a reactor used in the present step, the type and amount of the Pd-supported catalyst, and the type or amount of a solvent described later. In the case of using a fixed-bed reactor as the reactor, the amount of the titanosilicate catalyst used is adjusted such that the two catalysts (titanosilicate catalyst and Pd-supported catalyst) and the Pd-free carbon material are densely charged to the fixed-bed reactor. In the case of using a stirred tank as the reactor, it is preferred to form a slurry to an extent that the two catalysts (titanosilicate catalyst and Pd-supported catalyst) and the Pd-free carbon material can be stirred sufficiently in a solvent described later. For example, the total amount of the titanosilicate catalyst, the Pd-supported catalyst and the Pd-free carbon material is indicated in weight per kg of the solvent used and can be preferably in the range of 0.001 kg/kg to 0.2 kg/kg, more preferably in the range of 0.01 kg/kg to 0.1 kg/kg.

The mass ratio of Pd of the Pd-supported catalyst to titanosilicate catalyst (Pd/titanosilicate catalyst) is preferably 0.00001 to 1, more preferably 0.0001 to 0.1, and still more preferably 0.001 to 0.05.

Moreover, the weight ratio between the Pd-supported catalyst and the titanosilicate catalyst can be adjusted according to the ratio of their respective reaction activities. When the activity of the Pd-supported catalyst has deteriorated with age during the reaction, the Pd-supported catalyst may be added to the solvent for the reaction. When the activity of the titanosilicate catalyst has deteriorated with age during the reaction, the titanosilicate catalyst may be added to the solvent for the reaction.

As described above, the present reaction is caused in a liquid phase. For causing the present reaction in a liquid phase, a solvent is used in the present step. For the present reaction, water, an organic solvent or a mixed solvent of water and an organic solvent (hereinafter, referred to as a “water/organic solvent mixture”) can be used. Since the present reaction forms hydrogen peroxide in the reaction system, the water/organic solvent mixture is preferable from the viewpoint that the present step can be carried out more safely. Moreover, since a by-product water is formed during the course of formation of propylene oxide in the present reaction, the solvent in the liquid phase may become a water/organic solvent mixture with the progression of the present reaction even when only an organic solvent is used as the initial solvent.

Examples of the organic solvent that can be used in the present reaction include methanol, 1-propanol, 2-propanol, t-butanol, acetone, acetonitrile, toluene, 1,2-dichloroethane, t-butyl methyl ether and 1,4-dioxane. The organic solvent is preferably acetonitrile.

In the present reaction, an additive such as a polycyclic compound can also be allowed to coexist for suppressing a by-product propane. The use of such an additive can further improve hydrogen-based propylene oxide selectivity (hydrogen efficiency). Specifically, polycyclic compounds having 2 to 30 rings such as anthracene, tetracene, 9-methylanthracene, naphthalene and diphenyl ether (see e.g., International Publication No. WO2008-156205); polycyclic compounds such as triphenylphosphine, triphenylphosphine oxide, benzothiophene and dibenzothiophene (see e.g., International Publication No. WO99/52884); monocyclic quinoid compounds such as benzoquinone; condensed polycyclic aromatic compounds such as anthraquinone, 9,10-phenanthraquinone, benzoquinone and 2-ethylanthraquinone (see e.g., Japanese Patent Laid-Open No. 2008-106030); etc., are known as the additive. Of these additives, condensed polycyclic aromatic compounds having 2 to 30 rings are preferable. Moreover, among the condensed polycyclic aromatic compounds, anthraquinone is more preferable; and in the case of using such an additive, it is preferred to use an anthraquinone-containing additive. In the case of using the solvent in the present reaction, the additive may be dissolved in the solvent or may be undissolved; and however, for further getting the effect of the additive, it is preferred that the additive should be selected as one that can be dissolved in the solvent.

The amount of the additive is indicated in the amount of substance per kg of the solvent and is preferably in the range of 0.001 mmol/kg to 500 mmol/kg, more preferably in the range of 0.01 mmol/kg to 50 mmol/kg.

Moreover, in the present step, a salt containing ammonium ion, alkylammonium ion or alkylarylammonium ion (hereinafter, these salts are collectively referred to as an “ammonium-based salt”) may further be used. The use efficiency of hydrogen in the present reaction can be enhanced by allowing the ammonium-based salt to exist in the liquid phase. Examples of the ammonium-based salt can include: inorganic acid salts such as ammonium sulfate, ammonium hydrogen sulfate, ammonium hydrogen carbonate, ammonium phosphate, ammonium hydrogen phosphate, ammonium dihydrogen phosphate, ammonium hydrogen pyrophosphate, ammonium pyrophosphate, ammonium halide, and ammonium nitrate; and organic acid salts such as ammonium acetate (e.g., ammonium carboxylate). Examples of preferable ammonium-based salts include ammonium dihydrogen phosphate.

In the case of using the ammonium-based salt, the amount of the ammonium-based salt added is indicated in the amount of substance per kg of the solvent and is preferably in the range of 0.001 mmol/kg to 100 mmol/kg.

Examples of oxygen used in the present reaction include molecular oxygen such as oxygen gas. The oxygen gas may be oxygen gas produced by an inexpensive pressure swing method or may be, if necessary, highly pure oxygen gas produced by cryogenic separation or the like. Oxygen-containing gas (e.g., air) can also be used instead of pure oxygen gas.

Hydrogen gas is generally used as hydrogen used in the present reaction.

The oxygen and hydrogen gases used in the present reaction can also be diluted with a gas for dilution that does not inhibit the progression of the present reaction, and then subjected to the present step. Nitrogen, argon or carbon dioxide can be used as the gas for dilution. Moreover, organic gas such as methane, ethane and propane may be used as the gas for dilution unless separation from propylene oxide obtained after the present reaction becomes significantly difficult. The amount of the oxygen and hydrogen used and the concentration of the gas for dilution for diluting these gases can be adjusted according to the amount of substance of the propylene used or other conditions such as reaction scale.

The molar ratio between oxygen and hydrogen charged into the reactor is indicated in oxygen:hydrogen and is preferably in the range of 1:50 to 50:1, more preferably in the range of 1:5 to 5:1. It is preferred for safety that the molar ratio should be set such that the amount of hydrogen in a gas phase in the reactor of the present step was out of a range that causes the explosion of the hydrogen.

The amount of propylene in the present reaction is indicated in propylene:oxygen (molar ratio to the oxygen used) and is preferably in the range of 1:5 to 5:1. The present step may use a continuous reaction apparatus or may use a batch reaction apparatus; it is industrially preferred to use the continuous reaction apparatus; and it is preferred to continuously carry out the present reaction using the continuous reaction apparatus. In the case of continuously carrying out the present reaction, the partial pressure ratio can be controlled using the flow. amounts of oxygen, hydrogen and propylene supplied to the reactor.

The reactor used in the present step is available as a fixed-bed reactor, stirred tank, or the like, as described above, and examples thereof specifically include flow fixed-bed reactors and flow slurry-completely mixed reactors.

The reaction temperature of the present reaction is preferably in the range of 0° C. to 150° C., more preferably in the range of 40° C. to 90° C.

On the other hand, the reaction pressure of the present reaction is preferably in the range of 0.1 MPa to 20 MPa, more preferably in the range of 1 MPa to 10 MPa, in terms of gage pressure.

<Other Steps>

The reaction mixture taken out of the reactor through the present step contains by-products such as propylene glycol, in addition to the formed propylene oxide and unreacted residual propylene, hydrogen and oxygen. Moreover, a by-product propane may be contained, albeit slightly, and the solvent may be contained in the reaction mixture when the solvent is used in the present reaction. The propylene oxide of interest can be separated from the reaction mixture by purification means known in the art. Examples of the purification means include separation by distillation.

A production rate of propylene oxide when the reaction conducted in the presence of Pd-free carbon material is higher than that when the reaction conducted without Pd-free carbon material. Thus, according to the present invention, propylene oxide can be produced with a high reaction rate. Therefore, the present invention has the effect that not only can propylene oxide be produced with improved hydrogen efficiency, but also it becomes easier to separate and purify the propylene oxide from the reaction mixture.

### EXAMPLES

Hereinafter, the present invention will be described more specifically with reference to Examples.

The measurements in Examples were conducted in the following manner.

### <Elementary Analysis Method>

1. The contents of Ti (titanium) and Si (silicon) were determined by alkali fusion, dissolution in nitric acid, and ICP emission spectroscopy.
2. The contents of Pd (palladium) in Pd-supported catalyst was determined by microwave degradation and ICP emission spectroscopy.
3. The existence or absence of Pd in Pd free carbon material was determined by semiquantitative analysis based on fundamental parameter (FP) method, using fluorescence X-ray ZSX Primus II (Rigaku Corp.). Its measurement range was F to U.
Lower detection limit:<0.01% by weight

### <X-ray Powder Diffraction (XRD)>

The X-ray powder diffraction pattern of a sample was determined using the following apparatus and conditions:
Apparatus: RINT2500V manufactured by Rigaku Corp.

### Source: Cu Kα X-rays

Output: 40 kV-300 mA
Scan range: 2 θ=0.75 to 30°
Scan speed: 1°/min.

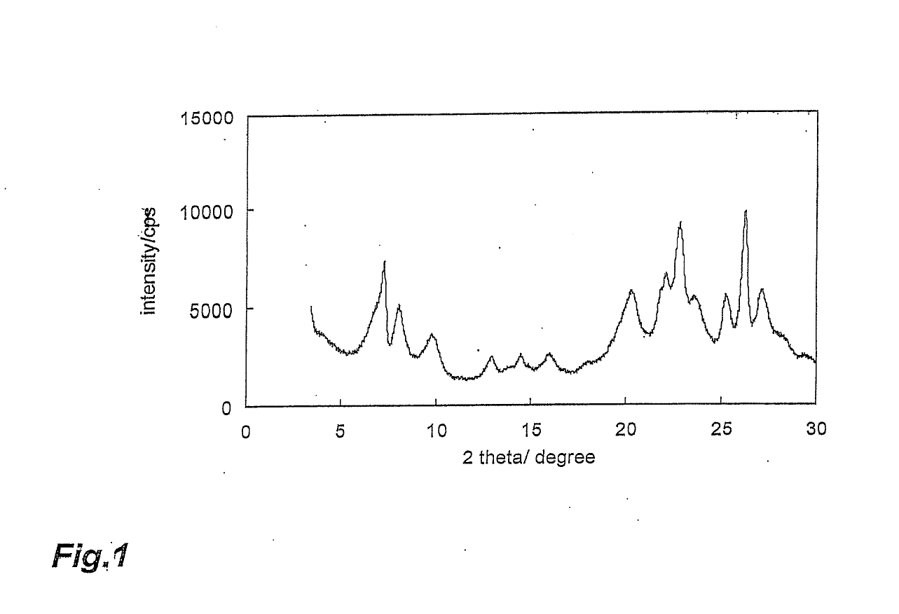

When the X-ray diffraction pattern was similar to that in FIG. 1 in EP1731515A1, the sample was determined to be a Ti-MWW precursor.

When the X-ray diffraction pattern was similar to that in FIG. 2 in EP1731515A1, the sample was determined to be Ti-MWW.

### <UV-Visible Absorption Spectrum (UV-Vis)>

A sample was well pulverized using an agate mortar and then pelletized (7 mmφ). The UV-visible absorption spectrum of this pellet was measured using the following apparatus and conditions:
Apparatus: diffuse reflection accessory (Praying Mantis manufactured by FIARRICK Scientific Products)
Attachment: UV-visible spectrophotometer (manufactured by JASCO Corp. (V-7100))
Pressure: atmospheric pressure
Measurement value: reflectance
Data capture time: 0.1 sec.
Band width: 2 nm
Measurement wavelength: 200 to 900 nm
Slit height: half-open
Data capture interval: 1 nm
Baseline correction (reference): BaSO₄ pellet (7 mmφ)

When the UV-visible absorption spectrum of a wavelength region of 200 nm to 400 nm had the greatest absorption peak in a wavelength region of 210 nm to 230 nm, the Ti containing silicate sample was determined to be titanosilicate.

### Preparation Example 1 [Preparation of Titanosilicate Catalyst (Titanosilicate A)]

In an autoclave, 899 g of piperidine, 2402 g of ion-exchanged water, 46 g of TBOT (tetra-n-butyl orthotitanate), 565 g of boric acid and 410 g of fumed silica (cab-o-sil M7D manufactured by Cabot Corp.) were charged at room temperature in an air atmosphere, and these were dissolved with stirring at this temperature in this atmosphere to prepare a gel. The obtained gel was aged for 1.5 hours, and then, the autoclave was tightly closed. The aged gel was further heated to 150° C. over 8 hours with stirring, then kept at this temperature for 120 hours for hydrothermal synthesis, and then cooled. The reaction product after the hydrothermal synthesis was a suspended solution. After filtration of the obtained suspended solution, the filter cake was washed with ion-exchanged water until the pH of the filtrate was 10.3.

Next, the filter cake was dried (drying temperature: 50° C.) until no decrease in weight was seen, to obtain 524 g of laminar compound. To 75 g of the obtained laminar compound, 3750 mL of 2 M aqueous nitric acid solution and 9.6 g of TBOT were added, and the mixture was then heated and heated for 20 hours with reflux kept. After cooling, filtration was performed, and the filter cake was washed with ion-exchanged water until the pH of the filtrate was around neutral, and vacuum-dried at 150° C. until no decrease in weight was observed. The obtained product was white powder. The above procedure was performed several times to obtain 120 g in total of white powder (hereinafter, referred to as a “white powder A1”).

The white powder A1 was calcined at 530° C. for 6 hours to obtain the white powder. The above procedure was performed several times to obtain 108 g in total of powder (hereinafter, referred to as a “white powder A2”).

In an autoclave, 300 g of piperidine, 600 g of ion-exchanged water and 80 g of the white powder A2 obtained above were charged at room temperature in an air atmosphere and dissolved with stirring at this temperature in this atmosphere to prepare a gel. The obtained gel was aged for 1.5 hours, and then, the autoclave was tightly closed. The aged gel was further heated to 160° C. over 4 hours with stirring, then kept at this temperature for 24 hours for hydrothermal synthesis. The reaction product after the hydrothermal synthesis was a suspended solution. After filtration of the obtained suspended solution, the filter cake was washed with ion-exchanged water until the pH of the filtrate was 9.6. Next, the filter cake was vacuum-dried at 150° C. until no decrease in weight was seen, to obtain white powder. Hereinafter, this white powder is referred to as a “white powder A3”.

In a three-neck glass flask, 175 mL of toluene and 4.0 g of the white powder A3 thus obtained were charged at room temperature in an air atmosphere and heated for 2 hours under reflux. After cooling, filtration was performed, and the filter cake was further washed with 500 mL of acetonitrile/ion-exchanged water=4/1 (weight ratio). The filter cake was further vacuum-dried at 150° C. until no decrease in weight was seen, to obtain 3.6 g of white powder. As a result of measuring the X-ray diffraction pattern (FIG. 1) and the UV-visible absorption spectrum (FIG. 2) of this white powder, this white powder was confirmed to be a Ti-MWW precursor (hereinafter this white powder is referred to as Ti-MWW precursor A).

Ti-MWW precursor A had a Ti content of 2.08% by weight and a Si content of 36.4% by weight. The calculated molar ratio of Ti/Si is from the Ti content and Si content was 0.034.

The Ti-MWW precursor A was subjected to activation treatment as described below.

The Ti-MWW precursor A (0.6 g) was added to 100 g of ion-exchanged water/acetonitrile=1/4 (weight ratio) solution containing 0.1% by weight of hydrogen peroxide, treated at room temperature for 1 hour, and filtered, and the filter cake was then washed with 500 mL of ion-exchanged water and suspended in 50 g of ion-exchanged water/acetonitrile=1/4 (weight ratio) solution. After the suspension, filtration and drying were performed to obtain a titanosilicate A.

### Preparation Example 2 [Preparation of Titanosilicate Catalyst (Titanosilicate B)]

In an autoclave, 898 g of piperidine, 2403 g of ion-exchanged water, 112 g of TBOT (tetra-n-butyl orthotitanate), 565 g of boric acid and 409 g of fumed silica (cab-o-sil M7D manufactured by Cabot Corp.) were charged at room temperature in an air atmosphere, and these were dissolved with stirring at this temperature in this atmosphere to prepare a gel. The obtained gel was aged for 1.5 hours, and then, the autoclave was tightly closed. The aged gel was further heated to 150° C. over 8 hours with stirring, then kept at this temperature for 120 hours for hydrothermal synthesis, and then cooled. The reaction product after the hydrothermal synthesis was a suspended solution. After filtration of the obtained suspended solution, the filter cake was washed with ion-exchanged water until the pH of the filtrate was around 10. Next, the filter cake was dried at 50° C. in a convection drying oven until no decrease in weight was seen, to obtain 517 g of laminar compound. To 75 g of the obtained laminar compound, 3750 mL of 2 M aqueous nitric acid solution was added, and the mixture was then heated for 20 hours with reflux kept under atmospheric pressure. After cooling, filtration was performed, and the filter cake was washed with ion-exchanged water until the pH of the filtrate was around neutral, and vacuum-dried at 150° C. until no decrease in weight was observed. The obtained product was white powder (hereinafter, referred to as a “white powder B1”).

The white powder B1 was calcined at 530° C. for 6 hours to obtain the white powder(hereinafter, referred to as a “white powder B2”). The above procedure was performed several times.

In an autoclave, 300 g of piperidine, 600 g of ion-exchanged water and 100 g of the white powder B2 obtained above were charged at room temperature in an air atmosphere and dissolved with stirring at this temperature in this atmosphere. The obtained mixture was aged for 1.5 hours, and then, the autoclave was tightly closed. The aged mixture was further heated to 150° C. over 4 hours with stirring, then controlled the temperature up to 160° C. The hydrothermal treatment was carried out for 1 day. The product after the hydrothermal treatment was a suspended solution. After filtration of the obtained suspended solution, the filter cake was washed with ion-exchanged water until the pH of the filtrate was around 9. Next, the filter cake was vacuum-dried at 150° C. until no decrease in weight was seen, to obtain white powder. The resulting white powder had a Ti content of 1.74% by weight and a Si content of 36.6% by weight. The calculated molar ratio of Ti/Si is from the Ti content and Si content was 0.028.

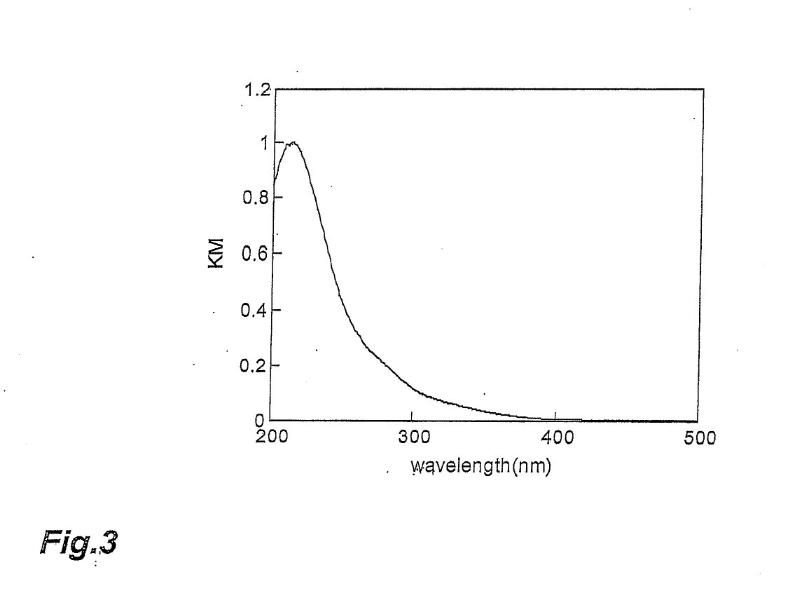

As a result of measuring the UV-visible absorption spectrum (FIG. 3) and , this white powder was a titanosilicate (hereinafter, referred to as a “titanosilicate B”).

### Preparation Example 3 [Preparation of Titanosilicate Catalyst (Titanosilicate C)]

White powder was prepared in the same method as in Preparation Example 2. Hereinafter, this white powder is referred to as a “Titanosilicate C.”

### Preparation Example 4 [Preparation of Pd-Supported Catalyst A (Pd/Active Carbon (AC) Catalyst)]

Active carbon (manufactured by Japan EnviroChemicals. ltd., Carborafin-6) washed in advance with 10 L of hot ion-exchanged water and dried in a nitrogen atmosphere at 300° C. for 6 hours was prepared. Moreover, a dispersion A was prepared from 0.3 mmol of colloidal palladium (manufactured by JGC C&C) (in terms of palladium) and ion-exchanged water.

In a 1-L eggplant-shaped flask, 3 g of the washed active carbon obtained above and 300 mL of ion-exchanged water were charged and stirred at room temperature in an air atmosphere. To the obtained suspension, 40 mL of the dispersion A was gradually added dropwise at room temperature in an air atmosphere. After the completion of the dropwise addition, the suspension was further stirred at this temperature in this atmosphere for 6 hours. After the completion of the stirring, the moisture was removed using a rotary evaporator, and the residue was vacuum-dried at 80° C. for 6 hours and then further heated in a nitrogen atmosphere at 300° C. for 6 hours to obtain a Pd/active carbon (AC) catalyst (hereinafter, referred to as a “Pd-supported catalyst A”).

### Preparation Example 5 [Preparation of Pd-Supported Catalyst B (Pd/Active Carbon (AC) Catalyst)]

Active carbon B for carrier of Pd-supported catalyst was prepared as follows. 20 g of active carbon (manufactured by Japan EnviroChemicals. ltd., TOKUSEI SHIRASAGI) was washed with 10 L of hot ion-exchanged water and then dried in a nitrogen atmosphere at 300° C. for 6 hours.

In a 1-L eggplant-shaped flask, 5 g of the active carbon B obtained above and 300 mL of ion-exchanged water were charged and stirred at room temperature in an air atmosphere, and then a dispersion prepared from 0.49 g of colloidal palladium solution (manufactured by JGC C&C) and ion-exchanged water was dropped gradually added dropwise into the flask with stirring. Herein, the colloidal palladium solution contained 3.1% by weight of Pd. After the completion of the dropwise addition, the suspension was further stirred at this temperature in this atmosphere for 6 hours. After the completion of the stirring, the moisture was removed using a rotary evaporator, and the residue was vacuum-dried at 80° C. for 6 hours and then further heated in a nitrogen atmosphere at 300° C. for 6 hours to obtain a Pd/active carbon (AC) catalyst (hereinafter, referred to as a “Pd-supported catalyst B”).

Pd content of Pd-supported catalyst B calculated by charged amount of material was 0.27% by weight.

### Preparation Example 6 [Preparation of Pd-Supported Catalyst C (Pd/Active Carbon (AC) Catalyst)]

Active carbon C for carrier of Pd-supported catalyst was prepared as follows. With 10 L of hot ion-exchanged water, 18 g of active carbon (manufactured by Japan EnviroChemicals. ltd., TOKUSEI SHIRASAGI) was washed.

After washing, the whole amount of the active carbon C was charged into a 1-L eggplant-shaped flask with 300 mL of ion-exchanged water at room temperature in an air atmosphere to obtain a mixture, and then the mixture was stirred.

After stirring, a dispersion prepared from 5.9 g of colloidal palladium solution (manufactured by JGC C&C) and ion-exchanged water was dropped gradually added dropwise into the flask with stirring. Herein, the colloidal palladium solution contained 3.1% by weight of Pd. After the completion of the dropwise addition, the suspension was further stirred at this temperature in this atmosphere for 6 hours. After the completion of the stirring, the moisture was removed using a rotary evaporator, and the residue was vacuum-dried at 80° C. for 6 hours and then further heated in a nitrogen atmosphere at 300° C. for 6 hours to obtain a Pd/active carbon (AC) catalyst (hereinafter, referred to as a “Pd-supported catalyst C”).

Pd content of Pd-supported catalyst C calculated by charged amount of material was 1.0% by weight.

### Preparation Example 7 [Preparation of Pd-free Carbon Material (Active Carbon A)]

Commercially available active carbon (manufactured by Japan EnviroChemicals. ltd., TOKUSEI SHIRASAGI) that does not substantially contain Pd was used for this preparation. It was confirmed by fluorescence X-ray analysis as mentioned above that the obtained active carbon has substantially no Pd. Twenty (20) g of this active carbon was washed with 1 L of ion-exchanged water and 10 L of hot ion-exchanged water in this order and heated in a nitrogen atmosphere at 300° C. for 6 hours to obtain active carbon A.

### Example 1 (Method for Producing Propylene Oxide)

A 0.5-L autoclave was used as a reactor. In the autoclave, 0.6 g of the titanosilicate A, 0.02 g of the Pd-supported catalyst A and 2 g of the active carbon A were charged. Continuous reaction was performed in which to this autoclave, source gas in which the volume ratio of propylene/oxygen/hydrogen/nitrogen was 8/11/4/77 and a solution containing anthraquinone dissolved in water/acetonitrile=1/4 (weight ratio) (anthraquinone concentration: 0.7 mmol/kg) were supplied at a supply rate of 16 L/hr and at a supply rate of 108 mL/hr, respectively, such that propylene, oxygen and hydrogen were reacted in the liquid phase in the reactor, from which the reaction mixture was then extracted via a filter. The reaction temperature was set to 60° C.; the pressure was set to 0.8 MPa (gage pressure); and the residence time of the supplied liquid in the reactor was set to 90 minutes.

The liquid and gas phases of the reaction product extracted after 5 hours into the reaction were analyzed by gas chromatography analysis and consequently determined to have propylene oxide produced at a rate of 4.06 mmol/hr, propylene oxide selectivity (molar amount of propylene oxide produced/(molar amount of propylene oxide produced+molar amount of propylene glycol produced+molar amount of propane produced)) of 93%, and by-product propane selectivity (molar amount of propane produced/(molar amount of propylene oxide produced+molar amount of propylene glycol produced+molar amount of propane produced)) of 3.2% and hydrogen efficiency (molar amount of propylene oxide produced/molar amount of hydrogen consumed) of 53%.

### Example 2 (Method for Producing Propylene Oxide)

First activation treatment of the titanosilicate C with hydrogen peroxide solution was subjected to as described below.

The titanosilicate C (2.28 g) was added to 100 g of ion-exchanged water/acetonitrile=1/4 (weight ratio) solution containing 0.1% by weight of hydrogen peroxide, treated at room temperature for 1 hour, and filtered, and the filter cake was then washed with ion-exchanged water for preparing an activated titanosilicate C. After the activation treatment, whole amount of the activated titanosilicate C described above was suspended in 100 ml of ion-exchanged water/acetonitrile=3/7 (weight ratio) solution for charging into a reactor.

Commercially available active carbon (manufactured by Japan EnviroChemicals. ltd., TOKUSEI SHIRASAGI) that does not substantially contain Pd was used as Pd-free carbon material for the reaction. Pd content of the active carbon was confirmed in the same manner as Preparation Example 7 and the active carbon has substantially no Pd.

A 0.3-L autoclave was used as a reactor. In the autoclave, 1.06 g of Pd-supported catalyst C and 2.1 g of the above Pd-free active carbon were charged into the autoclave and then the whole amount of the ion-exchanged water/acetonitrile solution including the activated titanosilicate C was charged into the autoclave.

Continuous reaction was performed in which to this autoclave, diluted source gas in which the volume ratio of oxygen/hydrogen/nitrogen was 3/4/93 and a solution containing anthraquinone and diammonium hydrogen phosphate dissolved in ion-exchanged water/acetonitrile=3/7 (weight ratio) (anthraquinone concentration: 0.7 mmol/kg, diammonium hydrogen phosphate concentration: 3.0 mmol/kg) and liquid propylene were supplied at a supply rate of at a supply rate of 263L/hr (0° C., 1 atm), 90 g/hr and 36 g/hr, such that propylene, oxygen and hydrogen were reacted in the liquid phase in the reactor, from which the reaction mixture was then extracted via a filter. The reaction temperature was set to 50° C.; the pressure was set to 4.0 MPa (gage pressure); and the residence time of the supplied solution in the reactor was set to 1 hour. Such reaction was continued for 4.5 hours before sampling.

During the reaction, the reaction mixture was adjusted to keep titanosilicate C at 2.28 g, Pd-supported catalyst at 1.06 g and Pd-free active carbon at 2.1 g in 90 g of its solvent.

The liquid and gas phases of the reaction product extracted after 4.5 hours into the reaction were analyzed by gas chromatography analysis and consequently determined to have propylene oxide produced at a rate of 162 mmol/hr, hydrogen consumed a rate of 281 mmol/hr, propylene oxide selectivity of 87%, and hydrogen efficiency of 58%.

### Comparative Example 1 (Method for Producing Propylene Oxide Without Use of Pd-Free Carbon Material)

The same procedure as in Example 1 was performed except that the active carbon A was not used, to perform production reaction of propylene oxide. The liquid and gas phases of the reaction product extracted after 5 hours into the reaction were analyzed by gas chromatography analysis and consequently determined to have propylene oxide produced at a rate of 3.50 mmol/hr, propylene oxide selectivity of 89%, and by-product propane selectivity of 5.3% and have hydrogen efficiency of 45%.

### Comparative Example 2 (Method for Producing Propylene Oxide Without Use of Pd-Free Carbon Material)

The same procedure as in Example 2 was performed except that the Pd-free active carbon was not charged into a reactor and that 2.28 g of titanosilicate B was used instead of titanosilicate C and that 3.17 g of Pd-supported catalyst B was used instead of Pd-supported catalyst C to perform production reaction of propylene oxide. The liquid and gas phases of the reaction product extracted after 4.5 hours into the reaction were analyzed by gas chromatography analysis and consequently determined to have propylene oxide produced at a rate of 146 mmol/hr, hydrogen consumed a rate of 305 mmol/hr, propylene oxide selectivity of 86%, and have hydrogen efficiency of 48%.

The present invention is exceedingly useful as a method for producing propylene oxide, which is an intermediate of various industrial materials.

1. A method for producing propylene oxide, comprising a step of reacting propylene, hydrogen and oxygen, in the presence of a Pd-supported catalyst, a titanosilicate catalyst and a Pd-free carbon material, in a liquid phase.

2. The method according to claim 1, wherein the Pd-supported catalyst consists of a carrier and Pd supported by the carrier, and the Pd-free carbon material does not form the carrier of the Pd-supported catalyst.

3. The method according to claim 1, wherein the Pd-supported catalyst comprises at least one carrier selected from the group consisting of silica, alumina, active carbon and carbon black.

4. The method according to claim 1, wherein the Pd-supported catalyst comprises a carrier selected from the group consisting of active carbon and carbon black.

5. The method according to claim 1, wherein the Pd-free carbon material is active carbon, carbon black or a mixture thereof.

6. The method according to claim 1, wherein the Pd-free carbon material is active carbon.

7. The method according to claim 1, wherein the step comprises reacting propylene, hydrogen and oxygen, further in the presence of a polycyclic compound having 2 to 30 rings, in a liquid phase.

8. The method according to claim 7, wherein the polycyclic compound is a condensed polycyclic aromatic compound.

9. The method according to claim 7, wherein the polycyclic compound comprises anthraquinone.
